(19) United States
(12) Patent Application Publication
(10) Pub. No.: US 2024/0067619 A1
(43) Pub. Date: Feb. 29, 2024

(54) METHOD FOR PREPARING CHIRAL MALEIMIDE DERIVATIVES USING ORGANIC CHIRAL CATALYST COMPOUNDS AND ECO-FRIENDLY SOLVENTS

(71) Applicant: KOREA UNIVERSITY RESEARCH AND BUSINESS FOUNDATION, Seoul (KR)

(72) Inventors: Hyeon Soo KIM, Seoul (KR); Jae Ho SHIM, Seoul (KR)

(21) Appl. No.: 18/209,894

(22) Filed: Jun. 14, 2023

(30) Foreign Application Priority Data
Aug. 12, 2022 (KR) 10-2022-0101243

(51) Int. Cl. C07D 307/94; B01J 31/02; C07D 207/40

(52) U.S. Cl. CPC C07D 307/94; B01J 31/0215; C07D 207/40; C07B 2200/09

(57) ABSTRACT

Provided are a method for preparing chiral maleimide derivatives using (R,R)-1,2-diphenylethylenediamine (DPEN)-based organic chiral catalyst compounds and water as an eco-friendly solvent, and the like. It is possible to prepare chiral maleimide derivatives having high enantioselectivity even with a small amount of catalyst in an excellent yield within a short time. In particular, the preparation method of the present disclosure can stabilize a transition state through an interfacial reaction between the catalyst and water. In addition, spironolactone derivatives are synthesized using chiral maleimide derivatives prepared according to the present disclosure to be usefully used for the treatment of edema control, heart failure, liver cirrhosis, electrolyte abnormalities, hypertension, etc.

### CROSS-REFERENCE TO RELATED APPLICATION

This application claims the benefit of Korean Patent Application No. 10-2022-0101243 filed on Aug. 12, 2022, in the Korean Intellectual Property Office, the entire disclosure of which is incorporated herein by reference for all purposes.

### BACKGROUND

### 1. Field of the Invention

The present disclosure relates to a method for preparing chiral maleimide derivatives using organic chiral catalyst compounds and eco-friendly solvents, a method for preparing non-natural spironolactone derivatives using the same, and the like.

### 2. Description of the Related Art

In understanding the basic properties of molecules, the spatial arrangement of atoms in a molecule has an important meaning. Over the last few decades, organic chemists have made many efforts to develop stereoselective organic reactions, and until recently, metal catalysts were used in significant amounts for asymmetric synthesis.

Metal catalysts exhibit high catalytic activity, but are expensive, and often unstable in reaction environments because metal ions contain air and moisture. In addition, when a metal catalyst is used, a small amount of metal may remain in products, which may cause environmental pollution. Therefore, in order to overcome these disadvantages, research on stereoselective synthesis using an organic catalyst has attracted attention. The organic catalyst generally contains carbons, hydrogens, nitrogens, and sulfurs, and is structurally different from a metal catalyst containing a metal core and ligands.

Heterocyclic compounds are one of large and various molecular fragment groups used by chemists for organic synthesis. The heterocyclic compounds are identified as special structures in medicinal chemistry and are known to have various pharmacological activities. Heterocyclic compounds with high enantioselectivity have been mainly synthesized through general organic synthesis reactions. However, conventional organic synthesis reactions require a large amount of metal or oxidizing agent, which are not economical and cause environmental problems by emitting by-products. In addition, since a reaction substrate is limited and other sensitive functional groups are not preserved during a reaction, it is difficult to synthesize (R)-type derivatives by conventional methods.

A Michael addition reaction is one of the most important carbon-carbon bond formation methods and is an important reaction used to synthesize a wide range of natural products or complex compounds that exhibit biological activities. The addition of α,α-disubstituted aldehyde to maleimide has a very important meaning, because not only the addition is a reaction to form a quaternary center, but also the products thereof are considered valuable synthetic targets and precursors of biologically interesting compounds.

Under this background, the present inventors developed a method for preparing maleimide derivatives in an eco-friendly manner with high yield and optical purity by introducing thiourea using (R,R)-1,2-diphenylethylenediamine (DPEN) as the basic skeleton of a chiral catalyst to apply aromatic group-substituted maleimide and aldehyde to a Michael addition reaction using hydrogen bond catalysis, and then completed the present disclosure.

### SUMMARY

An object of the present disclosure is to provide a method for preparing chiral maleimide derivatives through an asymmetric Michael addition reaction using a chiral thiourea catalyst.

Another object of the present disclosure is to provide chiral maleimide derivatives prepared by the preparation method.

Yet another object of the present disclosure is to provide a method for preparing chiral spironolactone derivatives using the chiral maleimide derivatives.

Still another object of the present disclosure is to provide chiral spironolactone derivatives prepared by the preparation method.

However, technical objects of the present disclosure are not limited to the aforementioned purpose and other objects which are not mentioned may be clearly understood by those skilled in the art from the following description.

According to an aspect, there is provided a method for preparing chiral maleimide derivatives including preparing a compound represented by Chemical Formula 3 by reacting a compound represented by Chemical Formula 1 with a compound represented by Chemical Formula 2 in water, in which a catalyst compound represented by Chemical Formula 4 is used in the reaction:

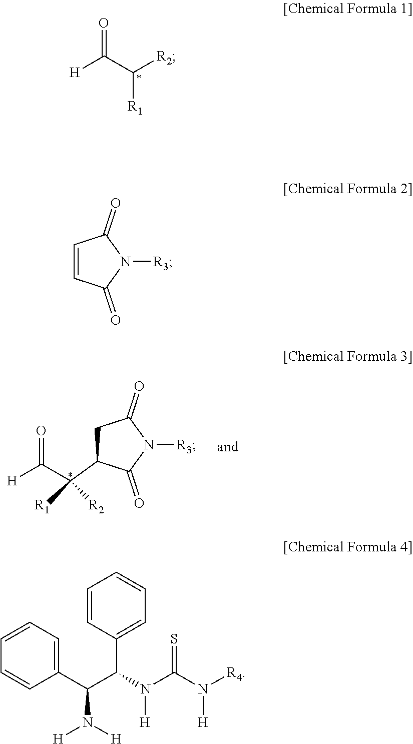

In Chemical Formulas 1 and 3, R₁ and R₂ are the same as or different from each other, and each independently hydrogen or a C₁-C₇ acyclic alkyl group (however, except when both R₁ and R₂ are hydrogens), in which the R₁ and R₂ may form a C₄-C₁₀ cycloalkyl group or heterocycloalkyl group together with carbons to which the R₁ and R₂ are attached and carbons marked with asterisks (*),
in Chemical Formulas 2 and 3, R₃ is a C₄-C₁₀ aryl group unsubstituted or substituted with at least one selected from the group consisting of a halogen group, a nitro group, a C₁-C₇ alkyl group, and combinations thereof, or hydrogen, and
in Chemical Formula 4, R₄ may be a C₄-C₁₀ aryl group unsubstituted or substituted with at least one selected from the group consisting of a halogen group, a cyano group, a nitro group, a trifluoromethyl group, a C₁-C₇ alkyl group, a C₁-C₇ alkoxy group, and combinations thereof.

As an embodiment of the present disclosure, the reaction may be an asymmetric Michael addition reaction.

As another embodiment of the present disclosure, the compound represented by Chemical Formula 1 may be at least one selected from compounds represented by Chemical Formulas 1-1 and 1-2:

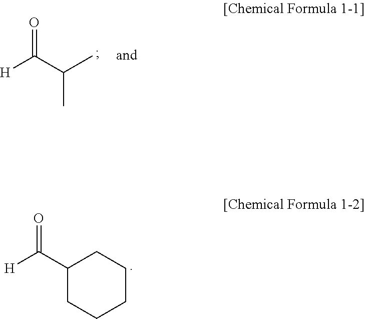

As another embodiment of the present disclosure, the compound represented by Chemical Formula 2 may be at least one selected from compounds represented by Chemical Formulas 2-1 to 2-5:

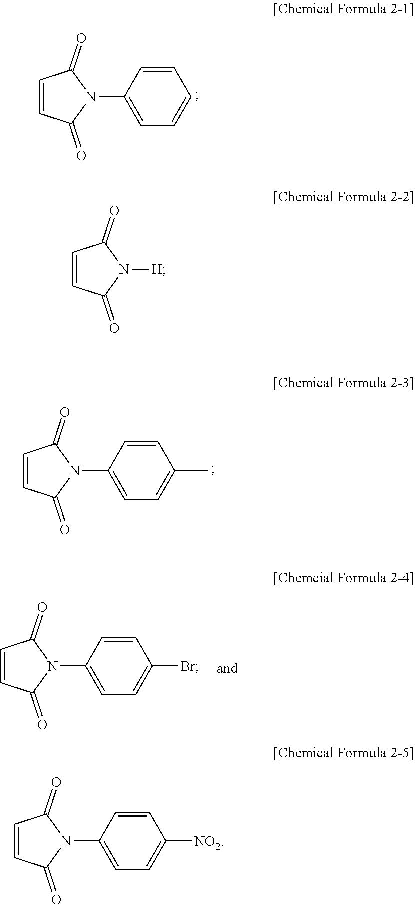

As another embodiment of the present disclosure, the compound represented by Chemical Formula 3 may be at least one selected from compounds represented by Chemical Formulas 3-1 to 3-6.

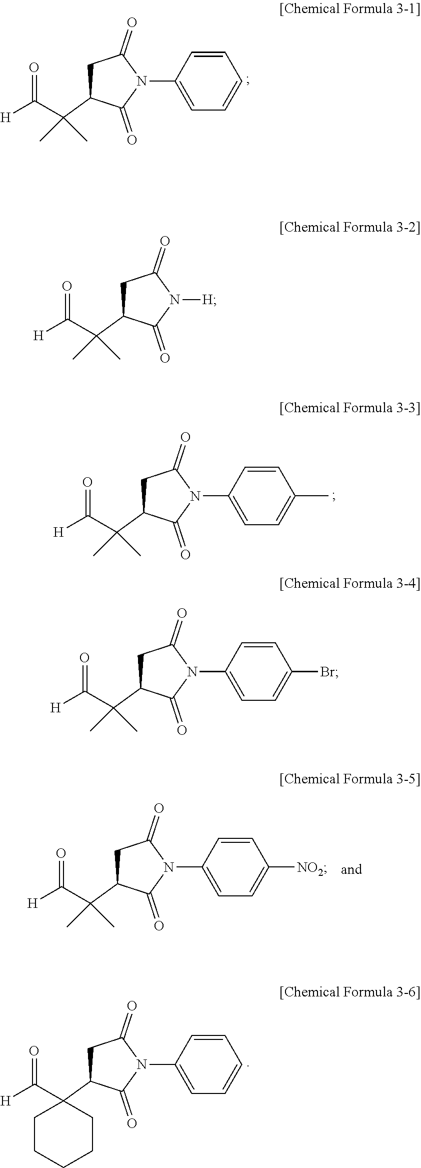

As another embodiment of the present disclosure, the compound represented by Chemical Formula 4 may be at least one selected from compounds represented by Chemical Formulas 4-1 to 4-6.

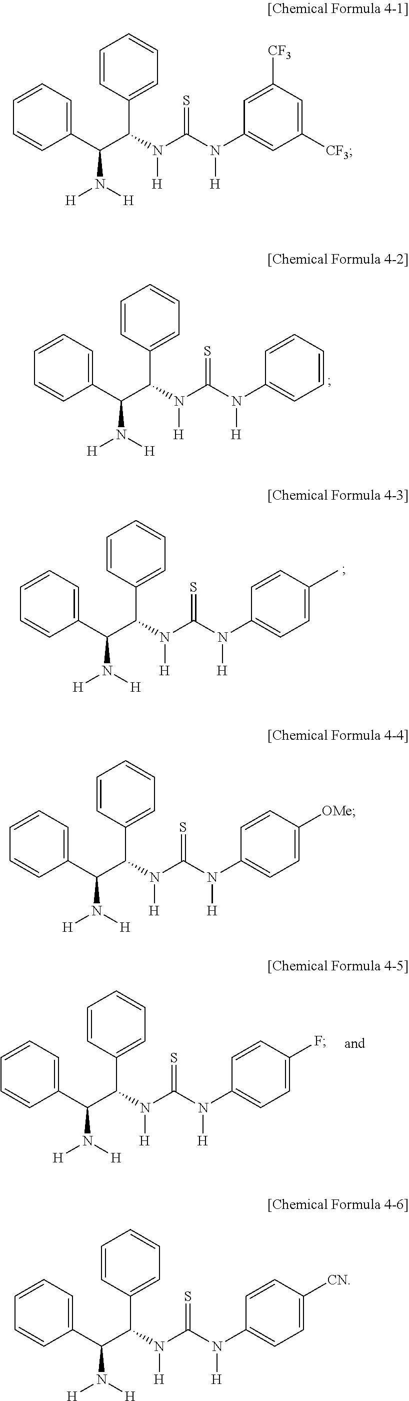

As another embodiment of the present disclosure, 10 mol % of trifluoroacetic acid, acetic acid, salicylic acid, or benzoic acid may be added and reacted together with the compound represented by Chemical Formula 1 and the compound represented by Chemical Formula 2.

As another embodiment of the present disclosure, the reaction may be performed in one reactor at room temperature.

As another embodiment of the present disclosure, the reaction may be completed within 1 to 15 hours.

As another embodiment of the present disclosure, 2 equiv. of the compound represented by Chemical Formula 1 and 1 equiv. of the compound represented by Chemical Formula 2 may react with each other. At this time, 0.005 to 1 mol %, preferably 0.01 mol % of the catalyst compound represented by Chemical Formula 4 may be added.

In addition, the present disclosure provides chiral maleimide derivatives prepared by the preparation method described above.

In addition, the present disclosure provides a method for preparing chiral spironolactone derivatives including preparing a compound represented by Chemical Formula 5 below from the chiral maleimide derivatives.

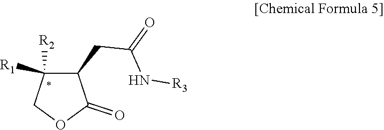

In Chemical Formula 5,
R₁ and R₂ are the same as or different from each other, and each independently hydrogen or a C₁-C₇ acyclic alkyl group (however, except when both R₁ and R₂ are hydrogens), in which the R₁ and R₂ may form a C₄-C₁₀ cycloalkyl group or heterocycloalkyl group together with carbons to which the R₁ and R₂ are attached and carbons marked with asterisks (*), and R₃ may be a C₄-C₁₀ aryl group unsubstituted or substituted with at least one selected from the group consisting of a halogen group, a nitro group, a C₁-C₇ alkyl group, and combinations thereof, or hydrogen.

As an embodiment of the present disclosure, in the chiral maleimide derivatives, when the R₁ and R₂ form a cyclohexyl group together with carbons to which the R₁ and R₂ are attached and carbons marked with asterisks (*), the spironolactone derivative may be a compound represented by Chemical Formula 5′.

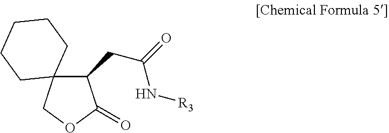

As another embodiment of the present disclosure, the chiral spironolactone derivatives may be prepared by adding BH₃·THF and BF3·Et₂O to the chiral maleimide derivatives. At this time, BH₃·THF and BF3·Et₂O may be sequentially added in CH₂Cl₂ as a solvent. Preferably, each BH₃·THF may be added and reacted for one day until the reaction is completed, and then BF₃·Et₂O may be added and further reacted for one day.

In addition, the present disclosure provides chiral spironolactone derivatives prepared by the preparation method described above.

According to embodiments, by the method for preparing the chiral maleimide derivatives, maleimide derivatives having enantioselectivity can be prepared through hydrophobic hydration using an (R,R)-1,2-diphenylethylenediamine (DPEN)-based thiourea catalyst. In addition, the catalyst may maintain yield and optical purity at high levels even when reused 4 or more times.

According to embodiments, the method for preparing the chiral maleimide derivatives is eco-friendly by using water as a solvent, and stabilizes a transition state through an interfacial reaction between the solvent and the catalyst compound even with a small amount of catalyst, thereby improving the yield and optical purity of the chiral maleimide derivatives as products and reducing the reaction time.

According to embodiments, since the chiral maleimide derivatives are used as intermediates in the synthesis of spironolactone and the like, which are used as physiologically active substances, spironolactone derivatives prepared using the chiral maleimide derivatives can be usefully used as a diuretic for the treatment of edema control, heart failure, liver cirrhosis, electrolyte abnormalities, and hypertension, and the like.

Effects of the chiral maleimide derivatives and the preparation method thereof according to the embodiment of the present disclosure are not limited to those mentioned above, and other effects not mentioned will be clearly understood by those skilled in the art from the following description.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1 illustrates Reaction Formula of a method for preparing chiral maleimide derivatives according to an embodiment of the present disclosure. Aldehyde (Chemical Formula 1) and maleimide (Chemical Formula 2) react together with a thiourea catalyst (Chemical Formula 4) in water (H₂O) to prepare a chiral maleimide derivative (Chemical Formula 3);

FIG. 2 illustrates a catalytic cycle of a Michael reaction including a proposed transition state;

FIG. 3 illustrates a catalytic mechanism proposed based on B3LYP/6-31G(d,p) calculations and a relative free energy diagram of an (R,R)-1,2-diphenylethylenediamine (DPEN)-thiourea-catalyzed enantioselective Michael reaction, in which the calculations were performed using water, CH₂Cl₂, THF, and the like under solvent+nH₂O conditions;

FIG. 4A and FIG. 4B illustrate results of nuclear magnetic resonance spectroscopy (NMR) to confirm hydrogen bonds between fluorine (F) atoms and hydrogen (H) atoms in a Michael addition reaction using an organic chiral catalyst 4-1 and water as a solvent. FIG. 4A illustrates an NMR result when there is water (D₂O) and FIG. 4B illustrates an NMR result when there is no water;

FIG. 5 illustrates a catalytic mechanism proposed based on B3LYP/6-31G(d,p) calculations and a thermal energy diagram of an (R,R)-1,2-diphenylethylenediamine (DPEN)-thiourea-catalyzed enantioselective Michael reaction, in which the calculations were performed under solvent+5H₂O conditions; and

FIG. 6 illustrates results of a recycling test of an asymmetric Michael addition reaction using a chiral (R,R)-1,2-diphenylethylenediamine (DPEN)-based thiourea catalyst.

### DETAILED DESCRIPTION

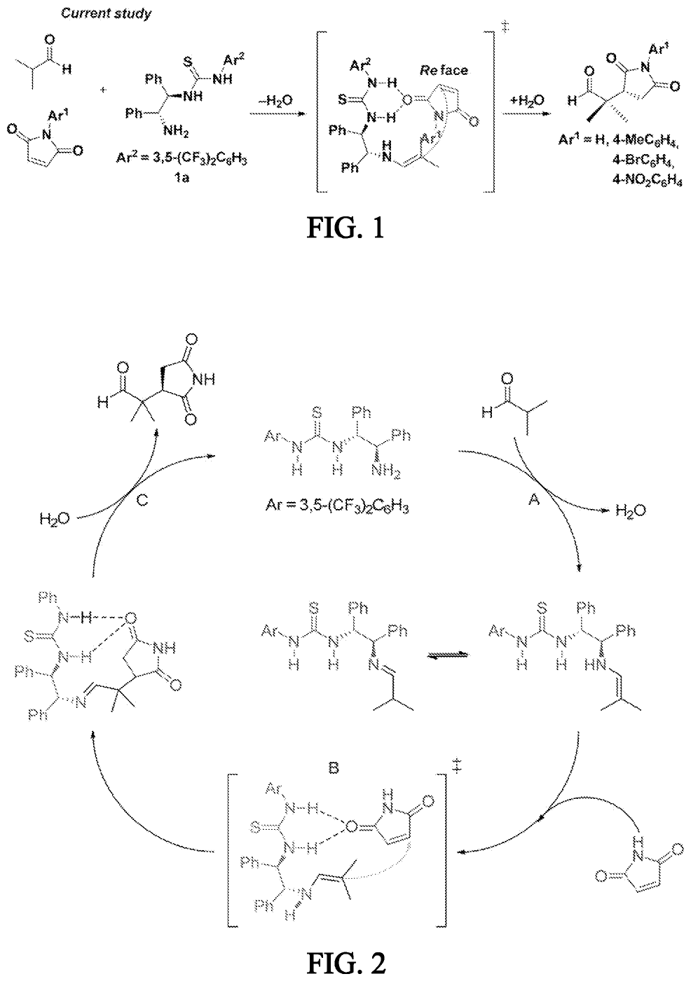

The present inventors applied an (R,R)-1,2-diphenylethylenediamine (DPEN)-based thiourea catalyst in water to an asymmetric Michael addition reaction of aldehyde and maleimide to prepare chiral maleimide derivatives having high enantioselectivity (94 to 99% ee) in a high yield of 97% or more in a short time even with a small amount (0.01 mol %) of catalyst (FIG. 1).

The present disclosure proceeds without metals and additives, can be performed in air, has a simple synthesis method, and is eco-friendly and economical because a required amount of catalyst is small. In addition, since the present disclosure shows excellent yield and optical purity even on a gram-scale, the chiral maleimide derivatives of the present disclosure and the chiral spironolactone derivatives prepared using the same can be used in various pharmaceutical synthesizes.

Accordingly, the present disclosure provides a method for preparing chiral maleimide derivatives, including preparing a compound represented by Chemical Formula 3 by reacting a compound represented by Chemical Formula 1 and a compound represented by Chemical Formula 2 in water using a catalyst compound represented by Chemical Formula 4:

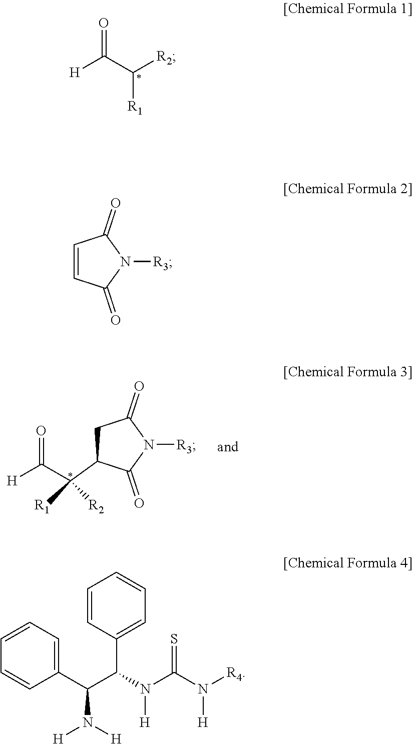

In Chemical Formulas 1 and 3, R₁ and R₂ are the same as or different from each other, and each independently hydrogen or a C₁-C₇ acyclic alkyl group (however, except when both R₁ and R₂ are hydrogens), in which the R₁ and R₂ may form a C₄-C₁₀ cycloalkyl group or heterocycloalkyl group together with carbons to which the R₁ and R₂ are attached and carbons marked with asterisks (*),
in Chemical Formulas 2 and 3, R₃ is a C₄-C₁₀ aryl group unsubstituted or substituted with at least one selected from the group consisting of a halogen group, a nitro group, a C₁-C₇ alkyl group, and combinations thereof, or hydrogen, and
in Chemical Formula 4, R₄ may be a C₄-C₁₀ aryl group unsubstituted or substituted with at least one selected from the group consisting of a halogen group, a cyano group, a nitro group, a trifluoromethyl group, a C₁-C₇ alkyl group, a C₁-C₇ alkoxy group, and combinations thereof.

In the present disclosure, the term “substitution” is a reaction in which atoms or atom groups included in molecules of a compound are replaced with other atoms or atom groups.

In the present disclosure, the term “acyclic alkyl group” refers to a group derived from straight-chain or branched-chain saturated aliphatic hydrocarbon having a specified number of carbon atoms and having at least one valency. Examples of such an alkyl group include methyl, ethyl, propyl, isopropyl, butyl, isobutyl, 2-butyl, 3-butyl, pentyl, n-hexyl, and the like, but are not limited thereto.

In the present disclosure, the term “cycloalkyl group” is also referred to as a cyclic alkyl group, and refers to a monovalent group having at least one saturated ring in which all ring members are carbons. Examples of such a cycloalkyl group include a cyclobutyl group, a cyclopentyl group, a cyclohexyl group, and the like, but are not limited thereto.

In the present disclosure, the term “heterocycloalkyl group” usually refers to saturated or unsaturated (but, not aromatic) cyclohydrocarbon, which may optionally be unsubstituted, monosubstituted or polysubstituted, and in its structure, at least one is selected from the group consisting of heteroatoms of N, O, or S.

In the present disclosure, the term “aryl group” refers to an unsaturated aromatic ring compound having 6 to 20 carbon atoms having a single ring (e.g., phenyl) or a plurality of condensed rings (e.g., naphthyl). Examples of such aryl include phenyl, naphthyl, and the like, but are not limited thereto.

In the present disclosure, the term “alkoxy group” refers to an atom group CₙH₂ₙ₊₁O⁻ formed by binding oxygen atoms to an alkyl group, and examples of such an alkoxy group include methoxy, ethoxy, propoxy, phthaloxy, and the like, but are not limited thereto.

In the present disclosure, the term “halogen group” refers to elements belonging to Group 17 of the periodic table, and may be fluorine (F), chloride (Cl), bromine (Br), iodine (I), or the like.

In addition, since the chiral maleimide derivatives of the present disclosure may be economically prepared because expensive metal catalysts are not used unlike conventional preparing methods and use water as an eco-friendly solvent under mild reaction conditions, the chiral spironolactone derivatives prepared using the chiral maleimide derivatives may be included in pharmaceutical compositions used for the treatment or prevention of various diseases.

The type of disease is not limited, but the chiral spironolactone derivatives of the present disclosure can be used as therapeutic agents for edema control, heart failure, liver cirrhosis, electrolyte abnormality, hypertension, etc., so that the present disclosure may provide a composition for the prevention or treatment of edema control, heart failure, liver cirrhosis, electrolyte abnormality, and hypertension, including derivatives represented by Chemical Formula 5 below as an active ingredient.

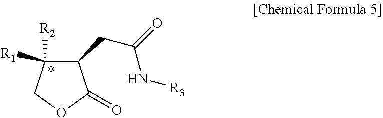

In Chemical Formula 5, R₁ and R₂ are the same as or different from each other, and each independently hydrogen or a C₁-C₇ acyclic alkyl group (however, except when both R₁ and R₂ are hydrogens), in which the R₁ and R₂ may form a C₄-C₁₀ cycloalkyl group or heterocycloalkyl group together with carbons to which the R₁ and R₂ are attached and carbons marked with asterisks (*), and R₃ may be a C₄-C₁₀ aryl group unsubstituted or substituted with at least one selected from the group consisting of a halogen group, a nitro group, a C₁-C₇ alkyl group, and combinations thereof, or hydrogen.

The pharmaceutical composition according to the present disclosure may include a pharmaceutically acceptable carrier in addition to the active ingredients. At this time, the pharmaceutically acceptable carrier is generally used in preparation and includes lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methylcellulose, methyl hydroxybenzoate, propyl hydroxybenzoate, talc, magnesium stearate, mineral oil, and the like, but is not limited thereto. Further, the pharmaceutical composition may further include a lubricant, a wetting agent, a sweetening agent, a flavoring agent, an emulsifying agent, a suspending agent, a preservative, and the like, in addition to the ingredients.

The pharmaceutical composition of the present disclosure may be administered orally or parenterally (e.g., applied intravenously, subcutaneously, intraperitoneally, or topically) according to a desired method, and a dose thereof varies depending on the condition and body weight of a patient, the severity of disease, a drug form, and route and time of administration, but may be appropriately selected by those skilled in the art.

The pharmaceutical composition of the present disclosure is administered in a pharmaceutically effective dose. The ‘pharmaceutically effective dose’ used herein refers to an amount enough to treat diseases at a reasonable benefit/risk ratio applicable to medical treatment. The effective dose level may be determined according to factors including the type and severity of a disease of a patient, the activity of a drug, the sensitivity to a drug, a time of administration, a route of administration, an excretion rate, duration of treatment, and simultaneously used drugs, and other factors well-known in the medical field. The pharmaceutical composition according to the present disclosure may be administered as an individual therapeutic agent or in combination with other therapeutic agents, and may be administered sequentially or simultaneously with conventional therapeutic agents, and may be administered singly or multiply. It is important to administer an amount capable of obtaining a maximum effect with a minimal amount without side effects by considering all the factors, which may be easily determined by those skilled in the art.

Specifically, the effective dose of the pharmaceutical composition of the present disclosure may vary depending on the age, sex, condition, and body weight of a patient, absorption rate, inactivity rate, and excretion rate of active ingredients in the body, a disease type, and combined drugs.

As another aspect of the present disclosure, the present disclosure provides a method for the prevention or treatment of edema control, heart failure, liver cirrhosis, electrolyte abnormality, and hypertension, including administering the pharmaceutical composition to a subject. The “subject” used herein refers to a subject in need of treatment or prevention for diseases, and more particularly, refers to mammals such as humans or non-human primates, mice, dogs, cats, horses, and cattle.

The terms used in the embodiments are used for the purpose of description only, and should not be construed to be limited. The singular expression includes the plural expression unless the context clearly dictates otherwise. In the present specification, it should be understood that term “comprising” or “having” indicates that a feature, a number, a step, an operation, a component, a part, or the combination thereof described in the specification is present, but does not exclude a possibility of presence or addition of one or more other features, numbers, steps, operations, components, parts or combinations thereof, in advance.

Unless otherwise contrarily defined, all terms used herein including technological or scientific terms have the same meanings as those generally understood by a person with ordinary skill in the art to which embodiments pertain. Terms which are defined in a generally used dictionary should be interpreted to have the same meaning as the meaning in the context of the related art, and are not interpreted as ideal or excessively formal meanings unless otherwise defined in the present application.

In describing the components of the embodiments of the present disclosure, terms including first, second, A, B, (a), (b), and the like may be used. These terms are just intended to distinguish the components from other components, and the terms do not limit the nature, sequence, or order of the components. When it is disclosed that any component is “connected”, “coupled”, or “linked” to other components, it should be understood that the component may be directly connected or linked to other components, but another component may be “connected”, “coupled”, or “linked” between the respective components.

Hereinafter, embodiments will be described in detail with reference to the accompanying drawings. However, since various modifications may be made to the embodiments, the scope of the present disclosure is not limited or restricted by these embodiments. It should be understood that all modifications, equivalents and substitutes for the embodiments are included in the scope of the present disclosure.

In addition, in the description with reference to the accompanying drawings, like components designate like reference numerals regardless of reference numerals and a duplicated description thereof will be omitted. In describing the embodiments, a detailed description of related known technologies will be omitted if it is determined that they unnecessarily make the gist of the embodiments unclear.

The present disclosure may have various modifications and various embodiments and specific embodiments will be illustrated in the drawings and described in detail in the detailed description. However, this does not limit the present disclosure within specific embodiments, and it should be understood that the present disclosure covers all the modifications, equivalents, and replacements within the idea and technical scope of the present disclosure. In the interest of clarity, not all details of the relevant art are described in detail in the present specification in so much as such details are not necessary to obtain a complete understanding of the present disclosure.

### EXAMPLE 1. Synthesis of Chiral Maleimide Derivatives of Present Disclosure

1.1. Instruments and Reagents

Optical rotation was measured using an automatic digital polarimeter, and Fourier transform infrared (FT-IR) spectra were recorded using a Nicolet 380 FT-IR spectrophotometer (Thermo Electron Corporation). Nuclear magnetic resonance (¹H NMR and ¹³C NMR) spectra were obtained using Varian Gemini 300 (300, 75 MHz) and Bruker Avance 500 (500, 125 MHz) using tetramethylsilane as an internal standard. Low-resolution mass spectrometry profiles were obtained using JEOL the MStation JMS-700. Chiral high-performance liquid chromatography (HPLC) analysis was performed using a Jasco LC-1500 series HPLC system. Toluene (CaH₂), tetrahydrofuran (THF) (Na, benzophenone), and CH₂Cl₂ (CaH₂) reaction solvents were purified and used. Reagents used in this study were obtained from Aldrich, Acros, Alfa, Sigma, Merck, Fluka, TCI, and Lancaster, and were purified or dried by known methods, if necessary. Merck's silica gel 60 (230 to 400 mesh) was used as a stationary phase in column chromatography.

10 1.2. Synthesis of N-mono-thiourea Catalyst

(R,R)-1.2-diphenylethylenediamine (DPEN, 0.5 g, 0.235 mmol) was dissolved in toluene (2.50 mL), and then the solution was added with isothiocyanate (0.35 mL, 0.235 mmol) and stirred at 0° C. for 1 hour, and then the reaction was terminated with distilled water. The mixture was extracted with ethyl acetate (20 mL×3 times), dehydrated with MgSO₄, filtrated, and concentrated under reduced pressure, and purified by column chromatography (SiO₂, CH₂Cl₂: n-hexane=1:2) to separate a product (Chemical Formula 4) (Reaction Formula 1).

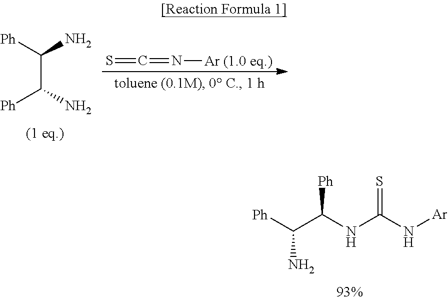

Non-limiting examples of the thiourea catalyst according to an embodiment of the present disclosure were as follows:

(1) 1-[(1R,2R)-2-Amino-1,2-diphenylethyl]-3-[3,5-bis(trifluoromethyl)phenyl]thiourea (Chemical Formula 4-1)

[α]_{D}²⁵ +13.5 (c 1.0, CH₃Cl); ¹H-NMR (500 MHz, DMSO-d6) δ 8.25 (s, 2H), 7.78 (s, 1H), 7.32-7.15 (m, 13H), 5.99 (d, J=3 Hz, 1H), 4.77 (d, J=3 Hz, 1H) ppm; ¹³C-NMR (125 MHz, DMSO-d6) δ 180.51, 143.26, 142.48, 130.82, 130.56, 128.51, 128.29 127.66, 127.55, 127.38, 124.78, 122.62, 121.34, 116.08, 63.66, 59.94 ppm; IR (KBr) 3305, 3032, 2963, 1652,1601, 1557, 1383, 1277, 1262, 803, 700 cm⁻¹; HRMS (FAB+) for C₂₃H₁₉F₆N₃S [M+H]⁺ Calcd: 484.1282, Found: 484.1254;

(2) 1-[(1R,2R)-2-Amino-1,2-diphenylethyl]-3-phenylthiourea (Chemical Formula 4-2)

[α]_{D}²⁰ =+62.0 (c=0.02, CH₂Cl₂); ¹H-NMR (300 MHz, CDCl₃) δ 7.76 (s, 1 H), 7.54-7.19 (m, 15 H), 5.54 (s, 1 H), 4.42 (d, 1 H, J=5 Hz), 1.35 (br s, 1 H); ¹³C-NMR (100 MHz, DMSO-d6) δ 182.09, 134.48, 133.93, 129.89, 128.70, 128.10, 127.91, 127.15, 126.94, 126.82, 126.74, 126.23, 125.59, 125.24, 122.98, 63.07,59.09; IR (KBr) 3287.86, 3027.84, 1521.63, 1241.99, 1072.28, 939.20, 698.13 cm⁻¹; HRMS (FAB+) for C₂₁H₂₂N₃S [M+H]⁺ Calcd: 348.4918, Found: 348.1534.

(3) 1-[(1R,2R)-2-Amino-1,2-diphenylethyl]-3-p-tolythiourea (Chemical Formula 4-3)

[α]_{D}²² +0.27 (c 1.00, CH₃Cl); ¹H-NMR (300 MHz, DMSO-d6) δ 9.79 (s, 1H), 7.13-7.38 (m, 15H), 5.50 (s, 1H), 4.32 (d, J=3 Hz, 1H), 2.27 (s, 3H) ppm; ¹³C-NMR (100 MHz, DMSOd6) δ 180.90, 143.72, 142.20, 137.14, 134.32, 129.86, 128.82 128.59, 127.68, 127.61, 127.46, 127.39, 123.97, 63.88, 60.00, 21.23 ppm; IR (KBr) 3301.69, 2861.98, 1889.99, 1527.42, 1342.28, 964.28, 701.99, 524.56 cm⁻¹; HRMS (FAB+) for C₂₂H₂₄N₃S [M+H]⁺ Calcd: 362.1691, Found: 362.2188

(4) 1-[(1R,2R)-2-Amino-1,2-diphenylethyl]-3-(4-methoxyphenyl)thiourea (Chemical Formula 4-4)

[α]_{D}²² +0.327 (c 1.00, CH₃Cl); 1H-NMR (300 MHz, DMSO-d6) δ 9.55 (s, 1H), 8.17 (s, 1H), 7.12-7.22 (m, 12H), 6.87 (d, J=6.0 Hz, 3H), 5.94 (s, 1H), 3.72 (s, 3H) ppm; ¹³C-NMR (100 MHz, DMSO-d6) δ 180.72, 156.54, 139.70, 131.67, 128.09, 127.97, 127.19, 125.61, 113.93, 62.38, 59.81. 55.24 ppm; IR (KBr) 3303.63, 3027.84, 1733.78, 1510.06, 1297.92, 1243.92, 1029.85, 831.21, 700.07, 568.93 cm⁻¹; HRMS (FAB+) for C₂₂H₂₄N₃OS [M+H]⁺ Calcd: 378.1640, Found: 378.1563

(5) 1-[(1R,2R)-2-Amino-1,2-diphenylethyl]-3-(4-fluorophenyl)thiourea (Chemical Formula 4-5)

[α]_{D}²² +0.132 (c 1.00, CH₃Cl); ¹H-NMR (300 MHz, DMSO-d6) δ 9.93 (s, 1H), 7.08-7.46 (m, 15H), 5.52 (s, 1H), 4.34 (d, J=3 Hz, 1H) ppm; ¹³C-NMR (100 MHz, DMSO-d6) δ 181.28, 160.71, 143.73, 142.10, 136.32, 128.79, 128.58, 127.70, 127.59, 127.49, 127.39, 125.79, 115.94, 115.71, 63.88, 60.06 ppm; IR (KBr) 3301.69, 3029.77, 1874.56, 1527.42, 1342.27, 1218.85, 840.85, 701.99 cm⁻¹; HRMS (FAB+) for C₂₁H₂₁FN₃S [M+H]⁺ Calcd: 366.1440, Found: 366.1440

(6) 1-[(1R,2R)-2-Amino-1,2-diphenylethyl]-3-(4-cyanophenyl)thiourea (Chemical Formula 4-6)

[α]_{D}²² +1.50 (c 1.00, CH₃Cl); ¹H-NMR (300 MHz, DMSO-d6) δ 10.50 (s, 1H), 7.07-7.88 (m, 15H), 5.56 (d, J=3 Hz, 1H), 4.39 (d, J=3 Hz, 1H) ppm; ¹³C-NMR (100 MHz, DMSO-d6) δ 180.47, 145.05, 143.34, 141.61, 133.36, 128.83, 128.64 127.74, 127.68, 127.59, 127.50, 121.37, 119.85, 105.11, 63.74, 60.04 ppm; IR (KBr) 3263.1, 2219.7, 1646.9, 1592.9, 1361.5, 1091.5 cm⁻¹; HRMS (FAB+) for C₂₂H₂₀N₄S [M+H]⁺ Calcd: 372.1487, Found: 372.1400

1.3. Asymmetric Michael Addition Reaction of Aldehyde and Maleimide

An N-mono-thiourea catalyst (Chemical Formula 4, 0.01 mol %) and maleimide (Chemical Formula 2, 2.88 mmol) were added in a reaction container at room temperature and dissolved in water (0.1 mL) under air conditions. Then, the mixture was added with aldehyde (Chemical Formula 1, 2 equiv.) and stirred for 10 to 13 hours. After completion of the reaction with distilled water, the mixture was extracted with ethyl acetate (0.3 mL×3 times), dehydrated with MgSO₄, filtrated and concentrated under reduced pressure, and purified with column chromatography (SiO₂, CH₂Cl₂: hexane=1:3) to separate a product (Chemical Formula 3).

Non-limiting examples of the chiral maleimide derivatives prepared by the asymmetric Michael addition reaction between aldehyde and maleimide according to an embodiment of the present disclosure were as follows:

(1) (R)-2-(2,5-Dioxo-1-phenylpyrrolidin-3-yl)-2-methylpropanal (2a, Chemical Formula 3-1)

[α]_{D}²⁵ +6.2 (c 0.2, CH₂Cl₂); ¹H NMR (300 MHz, CDCl₃) δ 9.51 (s, 1H), 7.26-7.50 (m, 5H), 3.14 (dd, J=6.0, 12 Hz, 1H), 2.96 (dd, J=9.0, 18 Hz, 1H), 2.60 (dd, J=6.0, 12 Hz, 1H), 1.32 (s, 3H), 1.27 (s, 3H) ppm; ¹³C NMR (100 MHz, CDCl₃) δ 203.0, 177.1, 175.0, 132.0, 129.4, 128.9, 126.7, 48.8, 45.2, 32.1, 20.6, 19.8 ppm; LRMS (EI+) Calcd. for [C₁₄H₁₅NO₃]⁺: 245, found: 245; HPLC [Chiralcel OD-H, hexane/2-propanol=75/25, flow rate=0.7 mL/min, λ=210 nm, retention times: (major) 38.8 min, (minor) 32.3 min].

(2) (R)-2-(2,5-Dioxopyrrolidin-3-yl)-2-methylpropanal (2b, Chemical Formula 3-2)

[α]_{D}²¹ −9.00 (c 0.2, CH₂Cl₂); ¹H NMR (300 MHz, CDCl₃) δ 9.49 (s, 1H), 8.57 (br. s, 1H), 3.09 (dd, J=6.0, 9.0 Hz, 1H), 2.85 (dd, J=12, 18 Hz, 1H), 2.51 (dd, J=6.0, 18 Hz, 1H), 1.26 (s, 3H), 1.24 (s, 3H) ppm; ¹³C NMR (100 MHz, DMSO) δ 185.4, 183.3, 182.8, 53.3, 48.8, 38.8, 29.5, 28.4 ppm; LRMS (EI+) Calcd. for [C₈H₁₁NO₃]⁺: 169, found: 169; HPLC [Chiralcel AD-H, hexane/2-propanol=85/15, flow rate=0.7 mL/min, λ=210 nm, retention times: (major) 25.3 min, (minor) 33.7 min].

(3) (R)-2-(2,5-Dioxo-1-p-tolylpyrrolidin-3-yl)-2-methylpropanal (2c, Chemical Formula 3-3)

[α]_{D}²¹ −6.5 (c 0.2, CH₂Cl₂); ¹H NMR (300 MHz, CDCl₃) δ 9.52 (s, 1H), 7.26 (t, 3H), 7.15 (d, J=9.0 Hz, 2H), 3.14 (dd, J=6.0, 9.0 Hz, 1H), 2.96 (dd, J=9.0, 18 Hz, 1H), 2.56-2.64 (dd, J=6.0, 18 Hz, 1H), 2.37 (s, 3H), 1.31 (s, 3H), 1.28 (s, 3H) ppm; ¹³C NMR (100 MHz, CDCl₃) δ 203.0, 177.2, 175.2, 139.0, 130.1, 129.3, 126.5, 48.7, 45.2, 32.0, 21.4, 20.5, 19.8 ppm; LRMS (EI+) Calcd. for [C₁₅H₁₇NO₃]⁺: 259, found: 259; HPLC [Chiralcel OD-H, hexane/2-propanol=75/25, flow rate=0.6 mL/min, λ=210 nm, retention times: (major) 37.7 min, (minor) 31.3 min].

(4) (R)-2-[1-(4-Bromophenyl)-2,5-dioxopyrrolidin-3-yl]-2-methylpropanal (2d, Chemical Formula 3-4)

[α]_{D}²⁰ +5.7 (c 0.2, CH₂Cl₂); ¹H NMR (300 MHz, CDCl₃) δ 9.48 (s, 1H), 7.60 (d, J=9.0 Hz, 2H), 7.19 (d, J=6.0 Hz, 2H), 3.11 (dd, J=6.0, 9.0 Hz, 1H), 2.97 (dd, J=9.0, 18 Hz, 1H), 2.60 (dd, J=6.0, 18 Hz, 1H), 1.36 (s, 3H), 1.28 (s, 3H) ppm; ¹³C NMR (100 MHz, CDCl₃) δ 203.0, 176.8, 174.6, 132.6, 131.0, 128.3, 122.8, 48.9, 45.1, 32.2, 20.7, 20.1 ppm; LRMS (EI+) Calcd. for [C₁₄H₁₄BrNO₃]⁺: 323, found: 323; HPLC [Chiralcel OD-H, hexane/2-propanol=75/25, flow rate=0.6 mL/min, λ=210 nm, retention times: (major) 58.6 min, (minor) 31.5 min].

(5) (R)-2-[1-(4-Nitrophenyl)-2,5-dioxopyrrolidin-3-yl]-2-methylpropanal (2e, Chemical Formula 3-5)

[α]_{D}²⁰ +2.7 (c 0.1, CH₂Cl₂); ¹H NMR (300 MHz, CDCl₃) δ 9.47 (s, 1H), 8.33 (d, J=9.0 Hz, 2H), 7.58 (d, J=9.0 Hz, 2H), 3.13 (dd, J=6.0, 12 Hz, 1H), 3.02 (dd, J=12, 18 Hz, 1H), 2.68 (dd, J=6.0, 18 Hz, 1H), 1.42 (s, 3H), 1.31 (s, 3H) ppm; ¹³C NMR (100 MHz, CDCl₃) δ 203.0, 176.5, 174.1, 147.2, 137.6, 127.3, 124.6, 49.2, 45.2, 32.4, 21.0, 20.5 ppm; LRMS (EI+) Calcd. for [C₁₄H₁₄N₂O₅]⁺: 290, found: 290; HPLC [Chiralcel OD-H, hexane/2-propanol=80/20, flow rate=1.0 mL/min, λ=210 nm, retention times: (major) 72.6 min, (minor) 44.4 min].

(6) (R)-1-(2,5-Dioxo-1-phenylpyrrolidin-3-yl)cyclohexanecarbaldehyde (2f, Chemical Formula 3-6)

[α]_{D}²⁰ +5.2 (c 0.1, CH₂Cl₂); ¹H NMR (300 MHz, CDCl₃) δ 9.54 (s, 1H), 7.26-7.50 (m, 5H), 3.25 (dd, J=6.0, 9.0 Hz, 1H), 2.88 (dd, J=9.0, 18 Hz, 1H), 2.67 (dd, J=6.0, 18 Hz, 1H), 1.82-2.04 (m, 3H), 1.53-1.64 (m, 7H) ppm; ¹³C NMR (100 MHz, CDCl₃) δ 204.9, 177.4, 175.2, 132.3, 129.5, 129.0, 127.0, 52.6, 43.0, 31.9, 29.0, 28.5, 25.5, 21.8, 21.6 ppm; LRMS (FAB+) Calcd. for [C₁₇H1₉NO₃]⁺: 285, found: 285; HPLC [Chiralcel OD-H, hexane/2-propanol=75/25, flow rate=1.0 mL/min, λ=210 nm, retention times: (major) 53.2 min, (minor) 42.4 min].

1.4. Gram-Scale Asymmetric Michael Addition Reaction of Aldehyde and Maleimide

An N-mono-thiourea catalyst (Chemical Formula 4, 0.01 mol %) and maleimide (Chemical Formula 2, 288.7 mmol) were added in a reaction container at room temperature and dissolved in water (10 mL) under air conditions. Then, the mixture was added with aldehyde (Chemical Formula 1, 2 equiv.) and stirred for 10 to 13 hours. After completion of the reaction with distilled water, the mixture was extracted with ethyl acetate (30 mL×3 times), dehydrated with MgSO₄, filtrated and concentrated under reduced pressure, and then purified by column chromatography (SiO₂, CH₂Cl₂: n-hexane=1:3) to separate a product (Chemical Formula 3). Finally, the catalyst was separated by changing the column chromatography condition (SiO₂, CH₂Cl₂: n-hexane=1:3).

### EXAMPLE 2. Synthesis of Chiral Spironolactone Derivatives of Present Disclosure

An N-mono-thiourea catalyst (Chemical Formula 4, 0.01 mol %) and N-phenyl maleimide (Chemical Formula 2-1, 288.7 mmol) were added in a reaction container at room temperature and dissolved in water (10 mL) under air conditions. Then, the mixture was added with cyclohexane carboxaldehyde (Chemical Formula 1-2, 2 equiv.) and stirred for 14 hours. After completion of the reaction with distilled water, the mixture was extracted with ethyl acetate (30 mL×3 times), dehydrated with MgSO₄, filtrated and concentrated under reduced pressure, and then purified by column chromatography (SiO₂, CH₂Cl₂: n-hexane=1:3) to obtain a product (Chemical Formula 3-6) in 98.6% yield (8.19 g) and 99% optical purity (ee). Finally, the catalyst was separated by changing the column chromatography condition (SiO₂, CH₂Cl₂: n-hexane=1:3).

8.19 g of the product (Chemical Formula 3-6) was diluted with CH₂Cl₂ (45 mL) and cooled to −20° C. The product was added with BH₃·THF (60 mL, 60 mmol), and then the reaction mixture was left while stirring and the reaction was heated to room temperature. After completion of reduction (24 to 48 hours, determined by TLC), BF₃·Et₂O (12 mL, 100 mmol) was added at −20° C. and the reactant was stirred at room temperature for 24 hours. The reaction mixture was dried under reduced pressure and the product was purified using column chromatography with petroleum ether (40 to 60° C.)/ethyl acetate (6:4) to obtain desired spironolactone (Chemical Formula 5-1) (Reaction Formula 2).

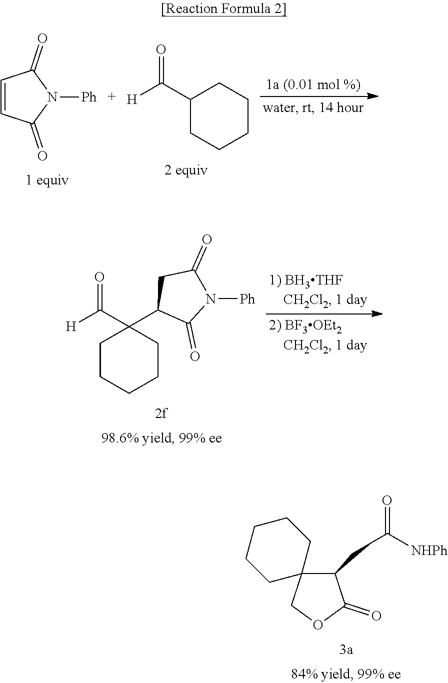

(R)-2-(3-Oxo-2-oxaspiro[4.5]decan-4-yl)-N-phenylacetamide (3a, Chemical Formula 5-1)

[α]_{D}²⁰ 4.2 (c 0.1, CH₂Cl₂); ¹H NMR (300 MHz, CDCl₃) δ 8.78 (s, 1H), 7.55 (d, J=6.0, Hz, 2H), 7.32 (t, J=6.0 Hz, 2H), 7.10 (t, J=9.0 Hz, 1H), 3.85 (m, 1H), 3.54 (m, 1H), 3.06 (dd, J=3.0, 9.0 Hz, 1H), 2.59 (dd, J=9.0, 15 Hz, 1H), 2.29 (dd, J=3.0, 15 Hz, 1H), 1.60 (m, 4H), 1.39 (m, 4H), 1.25 (m, 2H) ppm; ¹³C NMR (100 MHz, CDCl₃) δ 180.2, 170.0, 138.9, 129.5, 124.7, 120.5, 70.3, 47.3, 46.1, 33.2, 32.0, 28.9, 25.8, 23.1, 19.9 ppm; LRMS (FAB+) Calcd. for [C₁₇H₂₁NO₃]⁺: 287, found: 287; HPLC [Chiralcel OD-H, hexane/2-propanol=90/10, flow rate=1.0 mL/min, λ=254 nm, retention times: (major) 9.9 min, (minor) 14.9 min].

Since the spironolactons are diuretics known for a potential medicinal use, the chiral spironolactone derivatives of the present disclosure may be usefully used for the treatment of edema control, heart failure, cirrhosis, electrolyte abnormalities, hypertension, and the like.

### Experimental Example 1. Asymmetric Michael Addition Reaction Using Thiourea Catalyst

In order to examine an effect of the catalyst on the enantioselective Michael reaction of aldehyde and maleimide, the Michael addition reaction was performed by using DPEN as a basic backbone and a catalyst in which thiourea has replaced one of amines. At this time, the reaction was performed at room temperature using CH₂Cl₂ as a solvent. As a result, in the case of using a catalyst (Chemical Formula 4-1) substituted with a 3,5-bis(trifluoromethyl) group, the highest yield and stereoselectivity were exhibited.

1.1. Optimization of Additives and Solvents

The effect of acid additives was evaluated to further enhance the catalysis (Table 1). The acid additives activated the aldehyde to promote the catalytic function and the imine formation. In particular, the reaction yield slightly increased when benzoic acid was added (entry 4), and excellent yield and stereoselectivity were exhibited when the reaction solvent was changed to THF (entry 5).

However, when water was used as a solvent, the catalyst provided the highest reactivity and enantioselectivity with optimal yield and stereoselectivity even in the absence of weak acid additives, and the reaction time (2 to 36 times) and the catalyst amount (10 to 1000 times) were also dramatically reduced. In particular, in the case of entry 8 of Table 1, excellent yield and stereoselectivity were exhibited even with a supported catalyst amount of 0.01 mol %, which was 1/1000 of other solvents.

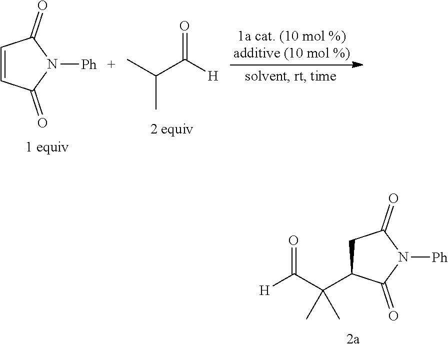

TABLE 1

| Entry | Time (h) | Additive | Solvent | Yield (%) ^{a} | ee (%) ^{b} |
|---|---|---|---|---|---|
| 1 | 24 | Trifluoro- | CH₂Cl₂ | 78 | 98 |
| | | acetic acid | | | |
| 2 | 24 | Acetic acid | CH₂Cl₂ | 83 | 94 |
| 3 | 24 | Salicylic | CH₂Cl₂ | 82 | 94 |
| | | acid | | | |
| 4 | 24 | Benzoic | Toluene | 78 | 99 |
| | | acid | | | |
| 5 | 24 | Benzoic | THF | 98 | 99 |
| | | acid | | | |
| 6 ^{c} | 0.67 | — | Water | 99 | 99 |
| 7 ^{d} | 6 | — | Water | 99 | 99 |
| 8 ^{e} | 12 | — | Water | 97 | 99 |

^{a} Separated yield.
^{b} Enantiomeric excess (ee) values were determined by chiral-phase high-performance liquid chromatography (HPLC) using an OD-H column.
Reactions were run with catalyst(1a) loading of
^{c} 1,
^{d} 0.1,
^{e} 0.01 mol %.

1.2. Reaction Depending on Type of Maleimide

The reactions between isobutyraldehyde and various maleimides were performed according to the conditions optimized in Example 1.1. The reaction time was the shortest when the maleimide was not substituted with a phenyl group (Chemical Formula 2-2), and maleimides substituted with toluene, bromophenyl, and nitrophenyl groups (Chemical

Formulas 2-3, 2-4, and 2-5) also showed excellent yield and optical purity (Table 2).

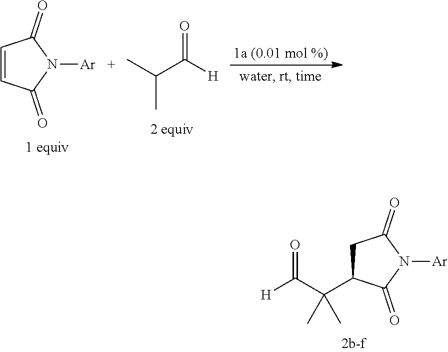

TABLE 2

| Entry | Ar | Time (h) | Yield (%) ^{a} | ee (%) ^{b} |
|---|---|---|---|---|
| 1 | H | 10 | 98 | 99 |
| 2 | 4-MeC₆H₄ | 12 | 99 | 99 |
| 3 | 4-BrC₆H₄ | 12 | 98 | 99 |
| 4 | 4-NO₂C₆H₄ | 12 | 97 | 99 |

^{a} Separated yield.
^{b} ee values were determined by chiral-phase HPLC using OD-H, AD-H, and AS-H columns.

### Experimental Example 2. Reaction Mechanism Inferred Through Expected Transition State

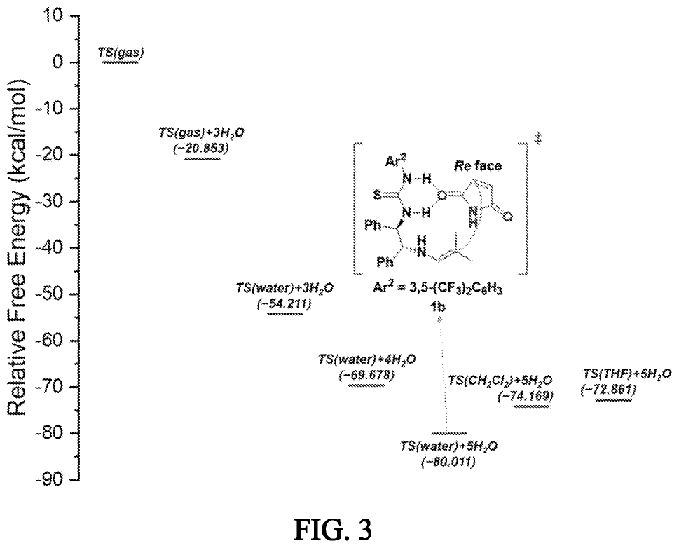

A mechanism of the Michael addition reaction between aldehyde and maleimide using a thiourea catalyst expected according to an embodiment of the present disclosure was illustrated in FIG. 2.

In step A, the thiourea catalyst reacted with isobutyraldehyde to form imine, and the imine formed enamine later. In addition, a thiourea portion of the catalyst and a ketone portion of the maleimide activated a maleimide electrophile through a hydrogen bond. Accordingly, a transition state including an electrophile activated in step B was formed, which can minimize steric hindrance of maleimide due to Re face attack. Finally, in step C, the catalyst and the product are separated through hydrolysis. According to the mechanism, the present disclosure may prepare an (R)-enantiomer as a main product.

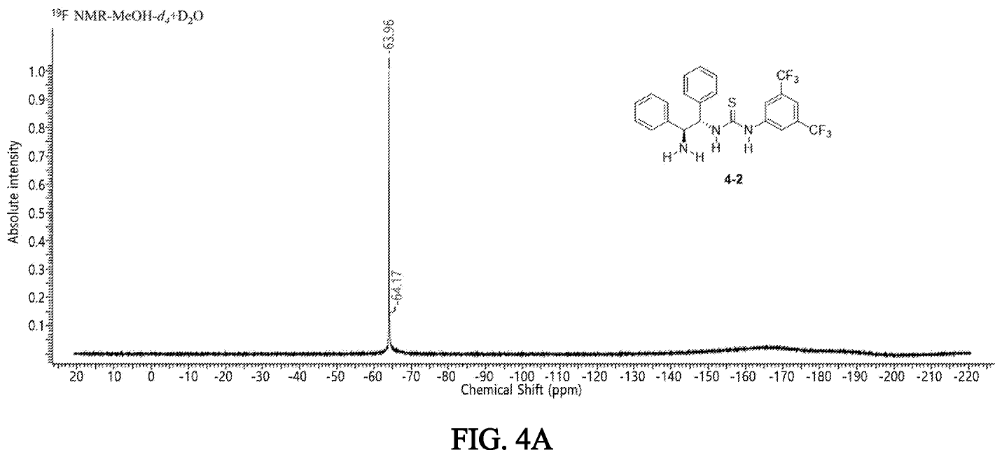

To more accurately predict a solvent effect of the catalyst, the relative free energy of the transition state during an interfacial reaction between fluorine atoms of a trifluoromethyl group of the thiourea catalyst (Chemical Formula 4-1) and water as a solvent was calculated as follows in an aqueous binary mixture (H₂O+solvent). When water was used as the solvent, the relative free energy of the transition state was calculated to be the lowest. As illustrated in FIG. 3, fluorine (F) atoms of the 3,5-bis(trifluoromethyl)phenyl group of the catalyst (Chemical Formula 4-1) interacted with hydrogen (H) atoms of the water solvent through hydrogen bonds, and the relative free energy was decreased as the number of hydrogen bonds in water was increased. That is, when water was used as the solvent in the Michael addition reaction, it means that the reactivity increased due to the stabilization of the relative energy and a hydrophobic effect of the hydration reaction.

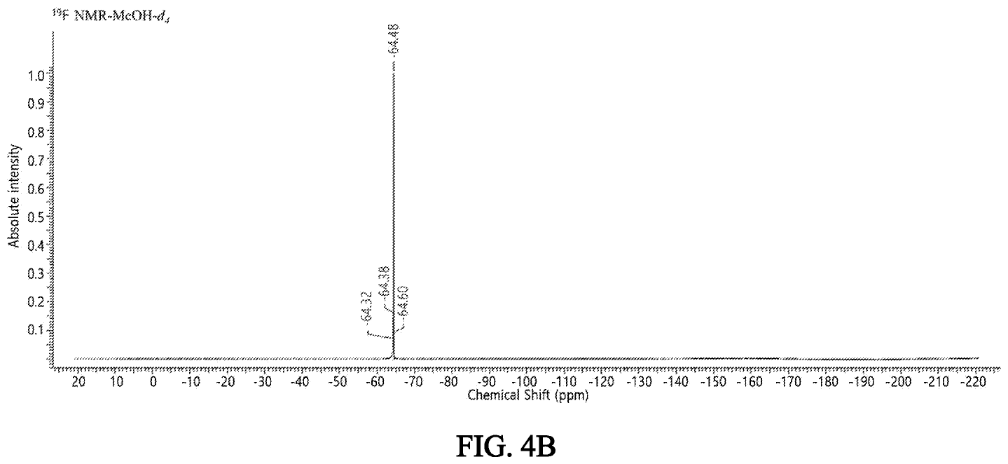

In addition, in order to prove through nuclear magnetic resonance spectroscopy (NMR) that the reaction proceeded through hydrogen bonds between fluorine (F) atoms and hydrogen (H) atoms in the reaction with the catalyst 4-1 when water was used as a solvent, as a result of measuring NMR by using D₂O instead of water, it was confirmed that the peaks of observing fluorine (¹⁹F) in the catalyst shifted depending on the presence or absence of D₂O (FIG. 4A and FIG. 4B). This suggests a possibility of forming hydrogen bonds between fluorine atoms and hydrogen atoms.

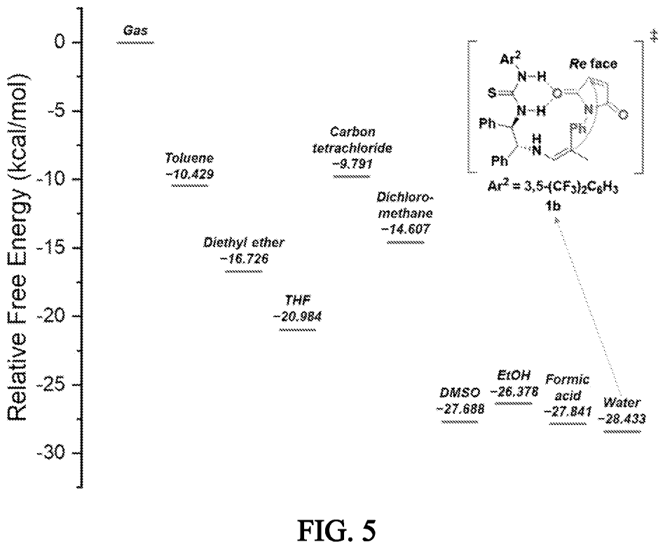

In general, hydrophobic non-polar solvents such as toluene provide excellent yield and stereoselectivity in the Michael addition reaction. However, since the solvent effect on the Michael addition reaction was confirmed in FIG. 3, a thermodynamic analysis was performed on the effect of water on the Michael addition reaction. Quantum calculations were performed to predict the relative free energy of the interfacial reaction system in the transition state of the catalyst (FIG. 5).

As a result of comparing the actual reaction results (Table 1) and quantum calculation results, toluene as a non-polar solvent showed the lowest reactivity, and tetrahydrofuran and CH₂Cl₂ showed similar reactivity in the calculated results. In particular, water exhibited the highest reactivity and stability beyond the results recorded for weak acids such as formic acid and dimethyl sulfoxide, and ethanol as polar hydrophilic solvents (FIG. 5).

### Experimental Example 3. Gram-Scale Asymmetric Michael Addition Reaction

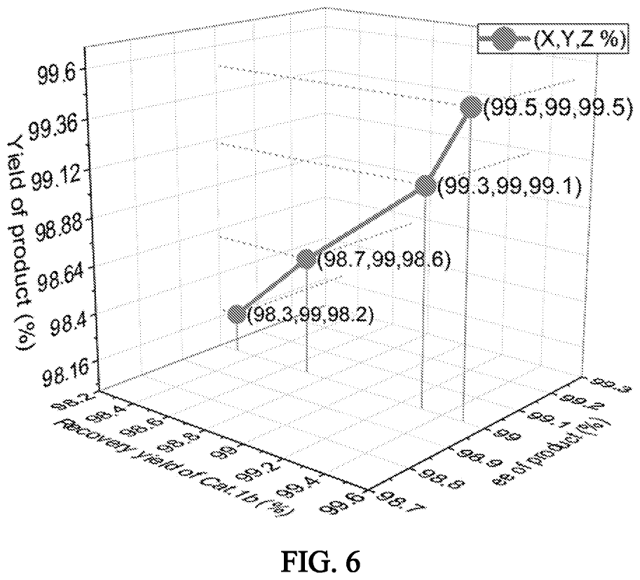

Recycling of the thiourea catalyst was evaluated (FIG. 6 and Reaction Formula 3). During 4 cycles of reuse, chiral maleimide derivatives produced through the Michael addition reaction maintained high yield (99.5 to 98.3%) and stereoselectivity (99%).

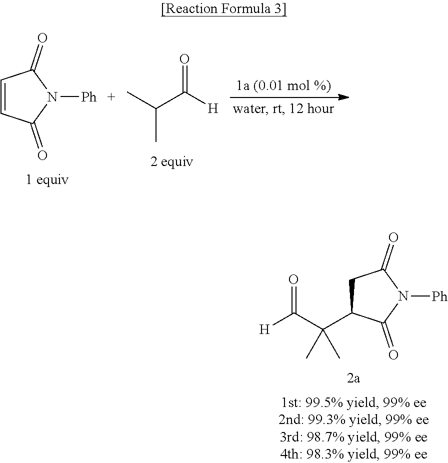

A gram-scale reaction of adding a cyclohexyl group to aldehyde was performed using the recovered catalyst. As in Example 2, through the Michael addition reaction of cyclohexane carboxaldehyde (Chemical Formula 1-2) and N-phenylmaleimide (Chemical Formula 2-1), (R)-1-(2,5-Dioxo-1-phenylpyrrolidin-3 -yl)cyclohexanecarbaldehyde (Chemical Formula 3-6) was produced, and then added with BH₃·THF and BF₃·Et₂O to produce (R)-2-(3-Oxo-2-oxaspiro[4.5]decan-4-yl)-N-phenylacetamide (Chemical Formula 5-1).

Therefore, according to an embodiment of the present disclosure, it is possible to prepare chiral maleimide derivatives with excellent yield (98.6%) and enantioselectivity (99%) even on a gram scale using an eco-friendly solvent and a recyclable catalyst. Based on this, it is possible to prepare chiral spironolactone derivatives with significant yield (84%) and enantioselectivity (99%).

As described above, although the embodiments have been described by the restricted drawings, various modifications and variations can be applied on the basis of the embodiments by those skilled in the art. For example, even if the described techniques are performed in a different order from the described method, and/or components such as a system, a structure, a device, a circuit, and the like described above are coupled or combined in a different form from the described method, or replaced or substituted by other components or equivalents, an appropriate result can be achieved.

Therefore, other implementations, other embodiments, and equivalents to the appended claims fall within the scope of the claims to be described below.

What is claimed is:

1. A method for preparing chiral maleimide derivatives comprising preparing a compound represented by Chemical Formula 3 by reacting a compound represented by Chemical Formula 1 with a compound represented by Chemical Formula 2 in water,
wherein a catalyst compound represented by Chemical Formula 4 is used in the reaction: 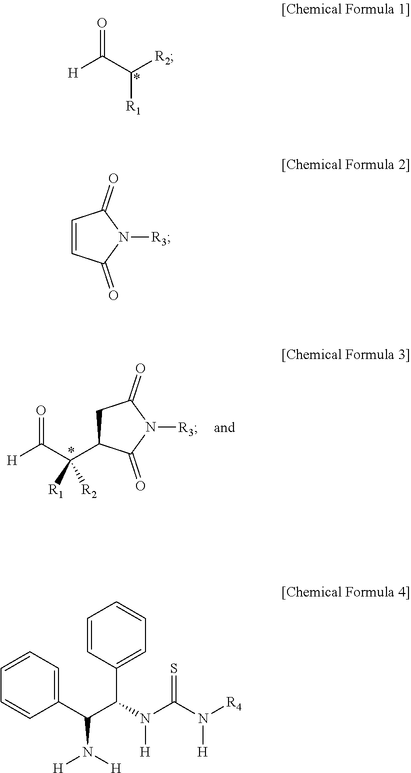
in Chemical Formulas 1 and 3,
R₁ and R₂ are the same as or different from each other, and each independently hydrogen or a C₁-C₇ acyclic alkyl group (however, except when both R₁ and R₂ are hydrogens), wherein the R₁ and R₂ may form a C₄-C₁₀ cycloalkyl group or heterocycloalkyl group together with carbons to which the R₁ and R₂ are attached and carbons marked with asterisks (*), and
in Chemical Formulas 2 and 3,
R₃ is a C₄-C₁₀ aryl group unsubstituted or substituted with at least one selected from the group consisting of a halogen group, a nitro group, a C₁-C₇ alkyl group, and combinations thereof, or hydrogen, and
in Chemical Formula 4,
R₄ is a C₄-C₁₀ aryl group unsubstituted or substituted with at least one selected from the group consisting of a halogen group, a cyano group, a nitro group, a trifluoromethyl group, a C₁-C₇ alkyl group, a C₁-C₇ alkoxy group, and combinations thereof.

2. The method for preparing the chiral maleimide derivatives of claim 1, wherein the reaction is an asymmetric Michael addition reaction.

3. The method for preparing the chiral maleimide derivatives of claim 1, wherein the compound represented by Chemical Formula 1 is at least one selected from the group consisting of compounds represented by Chemical Formulas 1-1 and 1-2:

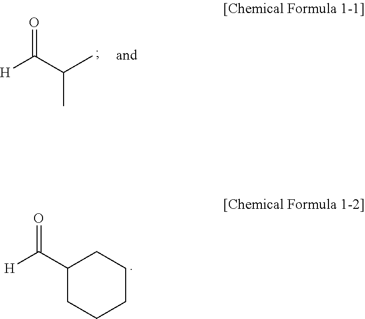

4. The method for preparing the chiral maleimide derivatives of claim 1, wherein the compound represented by Chemical Formula 2 is at least one selected from the group consisting of compounds represented by Chemical Formulas 2-1 to 2-5:

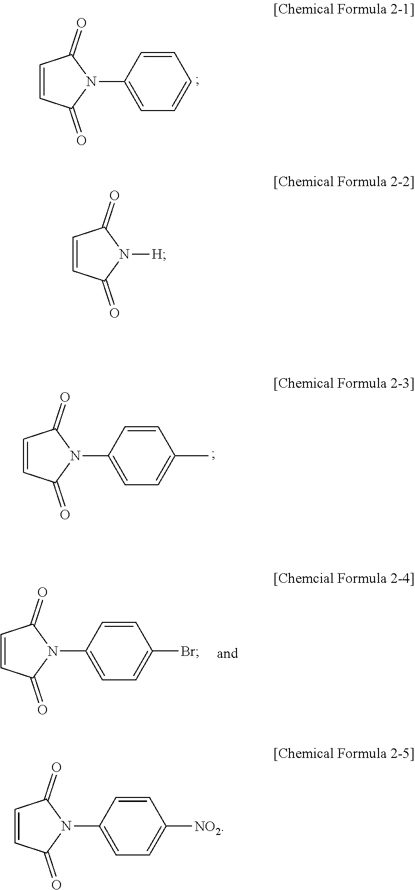

5. The method for preparing the chiral maleimide derivatives of claim 1, wherein the compound represented by Chemical Formula 3 is at least one selected from the group consisting of compounds represented by Chemical Formulas 3-1 to 3-6:

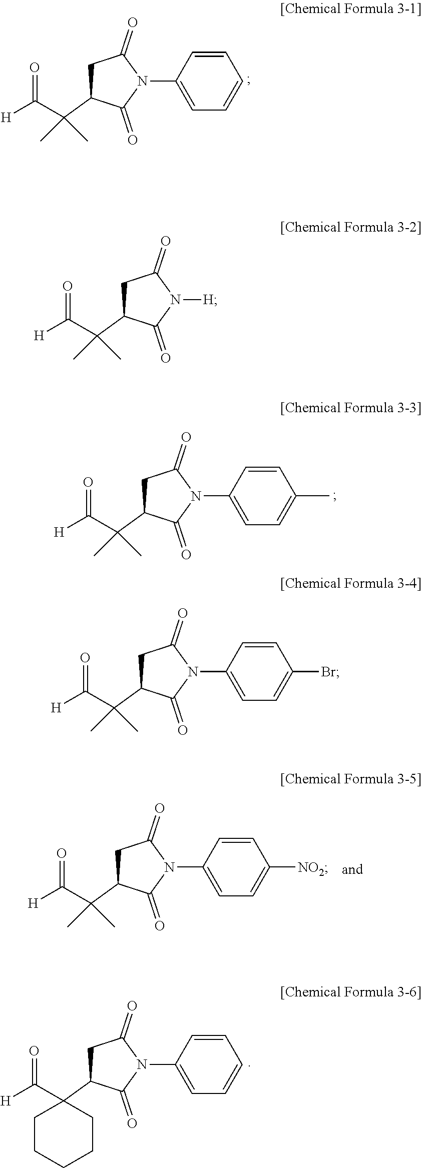

6. The method for preparing the chiral maleimide derivatives of claim 1, wherein the compound represented by Chemical Formula 4 is at least one selected from the group consisting of compounds represented by Chemical Formulas 4-1 to 4-6:

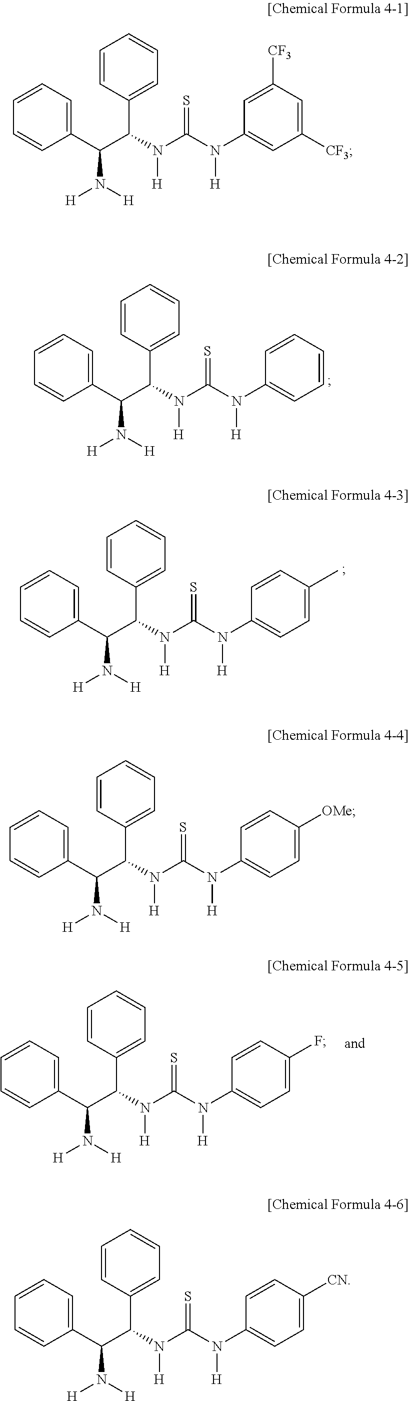

7. The method for preparing the chiral maleimide derivatives of claim 1, wherein trifluoroacetic acid, acetic acid, salicylic acid, or benzoic acid is further added and reacted together with the compound represented by Chemical Formula 1 and the compound represented by Chemical Formula 2.

8. The method for preparing the chiral maleimide derivatives of claim 1, wherein the reaction is performed in one reactor at room temperature.

9. The method for preparing the chiral maleimide derivatives of claim 1, wherein the reaction is completed within 1 to 15 hours.

10. The method for preparing the chiral maleimide derivatives of claim 1, wherein 0.005 to 1 mol % of the catalyst compound represented by Chemical Formula 4 is added.

11. Chiral maleimide derivatives prepared by the preparation method according to claim 1.

12. A method for preparing chiral spironolactone derivatives comprising:
(1) preparing a compound represented by Chemical Formula 3 by reacting a compound represented by Chemical Formula 1 with a compound represented by Chemical Formula 2 in water using a catalyst compound represented by Chemical Formula 4; and
(2) preparing a compound represented by Chemical Formula 5 below from the compound represented by Chemical Formula 3: 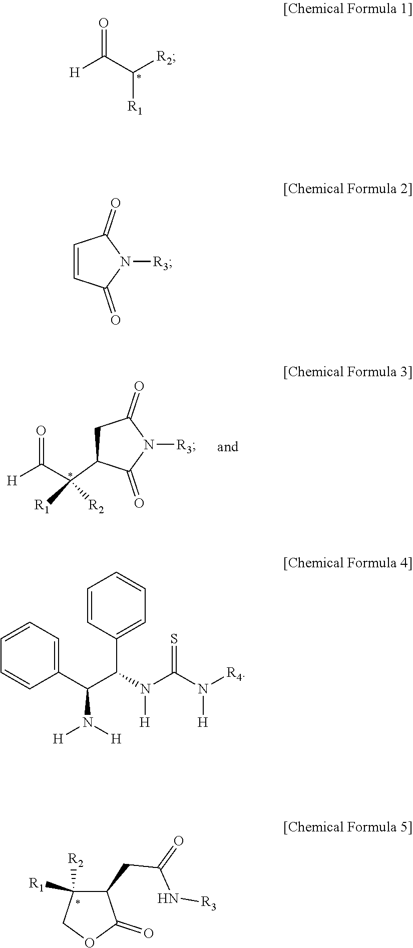
in Chemical Formulas 1, 3, and 5,
R₁ and R₂ are the same as or different from each other, and each independently hydrogen or a C₁-C₇ acyclic alkyl group (however, except when both R₁ and R₂ are hydrogens), wherein the R₁ and R₂ may form a C₄-C₁₀ cycloalkyl group or heterocycloalkyl group together with carbons to which the R₁ and R₂ are attached and carbons marked with asterisks (*), and
in Chemical Formulas 2, 3, and 5,
R₃ is a C₄-C₁₀ aryl group unsubstituted or substituted with at least one selected from the group consisting of a halogen group, a nitro group, a C₁-C₇ alkyl group, and combinations thereof, or hydrogen, and
in Chemical Formula 4,
R₄ is a C₄-C₁₀ aryl group unsubstituted or substituted with at least one selected from the group consisting of a halogen group, a cyano group, a nitro group, a trifluoromethyl group, a C₁-C₇ alkyl group, a C₁-C₇ alkoxy group, and combinations thereof.

13. The method for preparing the chiral spironolactone derivatives of claim 12, wherein when the R₁ and R₂ form a cyclohexyl group together with the carbons to which the R₁ and R₂ are attached and the carbons marked with asterisks (*), the compound represented by Chemical Formula 5 is a compound represented by Chemical Formula 5′ below:

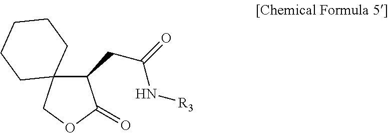

14. The method for preparing the chiral spironolactone derivatives of claim 12, wherein the compound represented by Chemical Formula 5 is added with BH₃·THF and BF₃·Et₂O.

15. Chiral spironolactone derivatives prepared by the preparation method according to claim 12.
